(19) United States
(12) Patent Application Publication
(10) Pub. No.: US 2026/0232236 A1
(43) Pub. Date: Aug. 13, 2026

(54) CANNULA INSERTION SYSTEM

(71) Applicant: Vitestro Holding B.V., Utrecht (NL)

(72) Inventors: Toon Olaf Overbeeke, Utrecht (NL); Brian Robert Joseph, 'S Gravenhage (NL); Johannes Gerhard Daniël Karssen, Utrecht (NL); Pascal Theodoor Wolkotte, Utrecht (NL); Johan Houdijn Beekhuis, Utrecht (NL); Tomas De Boer, Utrecht (NL)

(21) Appl. No.: 19/469,930

(22) PCT Filed: Mar. 27, 2024
(86) PCT No.: PCT/EP24/58393

§ 371 Date: Sep. 26, 2025

(30) Foreign Application Priority Data
Mar. 27, 2023 (NL) 2034438

(51) Int. Cl. A61B 5/15; A61B 5/00

(52) U.S. Cl. CPC A61B 5/150748; A61B 5/15003; A61B 5/150816; A61B 5/150824; A61B 5/742

(57) ABSTRACT

The invention provides a cannula insertion system for inserting a cannula into a body part of a patient, comprising: a cannula insertion device configured to insert the cannula into the body part of the patient, a body part support for supporting the body part during insertion of the cannula, and an indicator device to indicate a desired position of the body part on the body part support and/or a desired movement or direction of movement of the body part towards the desired position of the body part on the body part support.

The invention relates to a cannula insertion system for inserting a cannula into a body part of a patient.

US2008/0275396 A1 discloses an automated cannula insertion system for autonomously inserting a cannula into a blood vessel of a patient, the contents of which are herein incorporated by reference in its entirety.

The cannula insertion system of US2008/0275396 A1 comprises an acquisition module that allows for determining at least a location of a blood vessel underneath the skin and is enabled to determine a puncture location that is suitable for inserting a cannula into the blood vessel. The cannula insertion system comprises an actuator for moving and aligning the cannula to a determined position and to autonomously insert the cannula into the blood vessel for multiple purposes, such as blood withdrawal, intravenous medication and infusions.

To correctly insert a cannula, for example a needle, into the arm of a patient, the cannula insertion system may comprise an arm support configured to support the arm of the patient during insertion of the cannula into the arm of the patient.

A drawback of known arm supports is that the patient still has a relatively large freedom to position the arm with respect to the arm support. This may lead to an incorrect or suboptimal position of the arm with respect to the cannula insertion device. Such incorrect or suboptimal position of the arm with respect to the arm support may complicate or even prevent insertion of the cannula into the arm of the patient.

It is an object of the invention provide a cannula insertion system that allows to efficiently position a cannula at a target location into a body part of a patient. In particular, it is an object of the invention to provide an improved cannula insertion system that facilitates a proper positioning of the body part on the body part support.

The invention provides a cannula insertion system as claimed in claim 1.

According to the invention, the cannula insertion system comprises an indicator device to indicate a desired position of the body part on the body part support and/or a desired movement or direction of movement of the body part towards the desired position of the body part on the body part support.

The indicator device may be a passive device that indicates a desired position of the body part. The indicator device may for example comprise indicators that indicate the location where the wrist and/or elbow of the patient should be positioned.

In an embodiment, the indicator device comprises a light projection device arranged to project an indicator onto the body part and/or the body part support. The light project device can be used to indicate a desired position of the body part on the body part support. The light projection device may for example project a number of indicators such as light dots or other shapes which indicate the contour or specific features of the body part of a patient. The patient may be requested to position the body part such that all light dots or shapes are projected on the desired location of the body part of the patient. For example, the instruction may be that all projected dots are projected on the surface of the body part and not next to the body part. The projected indicator may also comprise one or more arrows. The direction of the arrow may indicate a desired direction of movement and/or the size of the arrow may indicate a desired distance to be moved.

When the body part is an arm, the indicator may be a projected light indicator indicating a desired position of a cubital fossa of arm. The cubital fossa of the arm of the patient, also referred to as antecubital fossa or elbow pit is often used as an area to select an insertion location, at which the cannula may be inserted into the arm of a patient. The projected light indicator may for instance be a triangular shape projected on the arm. The patient can be requested to align the triangular shape with the triangular shape of the cubital fossa.

In an alternative embodiment, the indicator may comprise a first projected light line indicating a desired position of a superior border of the cubital fossa and/or a second projected light line indicating a desired direction of the longitudinal axis of the arm.

The first light line can be used to locate the elbow of the patient at the desired location. The patient may be requested to position the superior border of the cubital fossa or elbow crease, aligned with the first light line. The second light line can be used to orientate the under arm with respect to the arm support by requesting the patient to align the second light line with the longitudinal axis of the under arm.

In yet another alternative embodiment, the indicator may comprise a projected light line indicating in transverse direction of the arm a desired position of a middle of the cubital fossa.

In further alternative embodiments other light lines or shapes may be used to help the patient to properly position the arm with respect to the arm support, or more generally another body part with respect to the body part support or another reference of the cannula insertion system.

The patient may be facilitated in placing the body part in the desired position by providing one or more marks on the body part. By requesting the patient to align the mark with the indicator projected by the light projection device, the body part can be brought into the desired position. The mark may for example be a sticker or a mark made with a marker pen. For example, a mark may be applied by an operator or a nurse at the location of the cubital fossa, and the patient may be requested to align this applied mark with an indicator projected by the light projection device.

The indicator device may be used to guide positioning of the body part, such as by indicating a desired position of the body part and/or one or more desired movements or directions of movement of the body part towards the desired position in one or more directions. These direction(s) may for example include an axial direction parallel to a longitudinal axis of the body part, e.g. in the direction of extension of an arm, a transverse direction, e.g. orthogonal to the longitudinal axis of the arm and/or a rotation, e.g. rotation of the arm about the longitudinal axis. Such rotation of the arm of the patient may for example be desirable when a first attempt to find a vein and/or a first attempt to draw blood was unsuccessful. By rotation of the arm another part of the arm, for example a side of the arm can be made accessible for the cannula insertion system.

Additional indicators, such as arrows, lines or shapes may be provided on the body part support to indicate a desired position of the body part with respect to the body part support. These indicators my include lights, such as LED's, or holographs. The indicator may for example also comprise a picture, drawing or print showing or representing a cubital fossa and/or an elbow, which picture, drawing or print is provided on the body part support to indicate a desired position of the cubital fossa and/or the elbow on the body part support.

In an embodiment, the cannula insertion system further comprises:
a sensor system arranged to determine an actual position of the body part with respect to the body part support or another reference of the cannula insertion system, and
a controller to compare the actual position with the desired position of the body part on the body part support in order to determine whether the body part is in the desired position and/or to determine the desired movement of the body part from the actual position to the desired position,
wherein the indicator device is connected to the controller and arranged to indicate whether the body part is in the desired position, or not, and/or to indicate the desired movement or direction of movement of the body part from the actual position to the desired position.

By providing a sensor system an actual position of the body part with respect to the body part support or the other reference of the cannula insertion system can be determined. When the actual position of the body part is known, the indicator device can be used to indicate whether the body part is in the desired position, or not, and/or to indicate the desired movement or direction of movement of the body part from the actual position to the desired position. In such an embodiment, the cannula insertion system can autonomously check whether the body part is positioned in the desired position without relying on confirmation of the patient or an operator.

In an embodiment, the light projection device may be arranged to project a first indicator representative for the desired position of the body part and a second indicator representative for the actual position of the body part, such that the body part can be brought into the desired position by predetermined alignment of the second indicator with respect to the first indicator.

By using two indicators that, when aligned, ensure that the body part is arranged in the desired position, a simple alignment instruction can be given to the patient that when properly followed will automatically lead to a correct positioning of the body part in the desired position on the body part support.

The sensor system may comprise a 2D camera system and/or a 3D camera system. Any camera, or sensor, that can be used to determine the actual position of the arm can be used. Advantageously, sensors that are also used to determine a suitable insertion location for the cannula to be inserted are used to avoid the need for additional cameras or sensors. In an embodiment, the sensor system is arranged to measure a 3D body part surface and/or to determine actual locations of predetermined features of the body part. A 3D body part surface and or the actual locations of predetermined features of the body part, such as the location of a wrist and cubital fossa of an arm of interest, can be beneficial to properly detect the actual position of the body part with respect to the body part support or another reference of the cannula insertion system.

In an embodiment, the indicator device comprises a light projection device arranged to project an indicator onto the body part, wherein the light projection device is configured to project at least two different colors, wherein the controller instructs the light projection device to project a first color of the at least two different colors when the body part is arranged in the desired position on the body part support and a second color of the at least two different colors when the body part is not arranged in the desired position on the body part support.

As an alternative or in addition to the use of indicators to indicate a desired location, a movement or a direction of movement, also different colours may be used to provide feedback to the patient on the positioning of the body part with respect to the body part support or another reference of the cannula insertion system.

In an embodiment, the body part is an arm and the body part support is an arm support.

If the body part is an arm, the controller may be arranged to determine a shape (e.g., pose and/or surface curvature) of the arm of the patient. On the basis of this shape (e.g., pose and/or surface curvature) a location of the insertion target area, for example a location of the cubital fossa, may be determined.

A contact sensor, e.g. an ultrasound probe, can be used to scan the shape (e.g., pose and/or surface curvature) of the arm of the patient, such as to determine a suitable puncture location. The determination of the surface curvature of the arm can, for example, prevent contact loss by anticipating the curvature of the arm during the scanning with the contact sensor.

The shape (e.g., pose and/or surface curvature) of the arm may be determined with any suitable method.

In an embodiment, images obtained by one or more cameras may be used to create a 3D model of the arm surface. On the 3D surface model a strip of points that run on top of the upper surface of the 3D model and along the center of the axial length of the patient's arm may be selected. Based on that collection of points, a straight line may be fitted between those points and the point that has the largest distance to the fitted line, indicates the location of the elbow crease and can be used to determine a location of the cubital fossa and/or another insertion target area.

In another method a complete 3D surface model of the upper surface of the arm can be used to find the shape (e.g., pose and/or surface curvature) of the arm. The 3D surface model can for example be fitted on a bone model or arm model to determine the curvature. On the basis of the determined curvature, a location of the cubital fossa and/or another insertion target area can be determined.

The actual position of the desired insertion target area, for example the cubital fossa, as determined on the basis of the pose and/or curvature of the arm can be compared with the desired position of the desired insertion target area, and the controller may provide instructions, for example projected indicators, to move the arm from the actual position to the desired position.

In an embodiment, the cannula insertion system comprises a hand grip to be held by the hand of the arm supported on the arm support. A hand grip can be used to facilitate the patient to keep his arm in the right orientation to carry out a cannula insertion on a suitable location on the arm.

In an embodiment, the hand grip is designed such that when the patient's hand is in the desired position gripping or otherwise engaging the hand grip, the patient's hand palm is faced upwards. By designing the hand grip such that the hand palm is faced upwards when the patient's hand is gripping the hand grip, the arm of the patient can be positioned in a beneficial position for carrying out a cannula insertion at a suitable location of the arm, for example at or close to the cubital fossa.

The hand grip may be a movable hand grip to allow the arm support to be adjusted to different arm lengths, while at the same time the elbow of the patient is arranged at substantially the same location. A hand grip locking device may be provided to lock the hand grip in a desired position. For example, the hand grip may be movable axially (e.g., generally in the direction of the length of the patient's arm) and/or transversely (e.g., generally orthogonal to the length of the patient's arm, from left side to right side of the patient's arm).

In an embodiment, the indicator device comprises a tactile feedback device to guide positioning of the body part on the body part support, such as by indicating the desired position of the body part on the body part support and/or the desired movement of the body part towards the desired position of the body part on the body part support.

In addition or as an alternative to projected light indicators, a tactile feedback device may be used to indicate to a patient a desired movement or desired direction of movement of the body part. The tactile feedback device may comprise one or more tactile feedback elements that can be used to provide tactile feedback to the patient with respect to the positioning of the body part. The tactile feedback elements may move, press, and/or vibrate to indicate an intended direction of movement. The tactile feedback elements may for example be pins or a hand grip, or other feedback elements.

In an embodiment, tactile feedback elements, such as pins, may be arranged at opposite sides (e.g., opposite lateral sides) of a body part and slowly move towards each other until they reach positions that limit the desired position of the body part. Alternatively, one or more tactile feedback elements on one side of the body part may remain stationary while one or more tactile feedback elements on an opposite side of the body part may move until tactile feedback elements on both sides of the body part are at positions that limit the desired position of the body part. If one of the tactile feedback elements contacts the body part and it is desired that the body part is moved by the patient the respective tactile feedback element may press or vibrate to indicate the desired movement of the body part.

In an embodiment, the cannula insertion system comprises a body part fixation device to fixate the body part on the body part support.

In order to position and hold an arm in a desired position a fixation device may be provided to fixate the arm at the desired position. For example, when the body part is an arm, the arm support may be provided with a wrist support fixation device to ensure the wrist will remain in its position on the arm support. Similarly the tourniquet device used to pinch the upper arm can also be used to fixate the upper arm on the arm support.

In an embodiment, the body part support comprises an actuation device to actuate the body part fixation device to move the fixated body part into a desired position.

When the arm support is provided as a combination of an elbow support and a wrist support that are movable with respect to each other by at least one actuation device, this relative movement can be used to move the arm of a patient to a desired position. The wrist support may comprise a wrist fixation device to fixate the wrist on the wrist support. Correspondingly, a tourniquet device can be used to fix the elbow on the elbow support. A wrist support actuation device can be provided to move the wrist support in a desired direction and/or an elbow support actuation device can be provided to move the elbow support in a desired direction.

By moving the wrist support, while the wrist is fixated on the wrist support, in an axial direction the arm can be moved or otherwise guided to a desired axial position with respect to the elbow support. Simultaneously or subsequently, the arm can be moved or otherwise guided to a desired transverse position by moving the elbow support, while the elbow is fixated on the elbow support, in a transverse direction with respect to the wrist support. These movements of the wrist support and elbow support work as an indicator device to the patient as the patient will be urged to follow the movements. Other combinations of movements of the wrist support and elbow support can also be made to bring the arm in the desired position.

In an embodiment, the cannula insertion device comprises a display device to display instructions and/or information to the patient.

In an embodiment, the cannula insertion system comprises a light projection device arranged to project instructions and/or information on the body part.

According to another aspect of the invention there is provided a cannula insertion system to insert a cannula into a body part of a patient, comprising:
a cannula insertion device configured to insert the cannula into the body part of the patient,
a body part support for supporting the body part during insertion of the cannula, and
a light projection device arranged to project instructions and/or information on the body part.

It has been found that patients may be focused on the body part, for example arm, in which the cannula is inserted during the cannula insertion process. Therefore, instructions provided on a display device may not be noticed. It is proposed to use a light projection device to project instructions and/or information on this body part of the patient. These instructions and/or this information, that may or may not be simultaneously be displayed on a display device or otherwise provided to the patient will more likely be noticed.

Instructions that may be displayed to the patient are for example instructions to position, reposition and/or remove the body part, for example the arm, with respect to the body part support, to keep the body part in a certain position with respect to the body part support and/or to twist a wrist to hold a hand grip in the desired way with the hand palm upwards. Information to be displayed may for example be the expected time needed to complete the cannula insertion procedure, or the phase of the procedure (insertion location detection, cannula insertion, blood withdrawal, bandage application, etc.), or any other type of information.

According to yet another aspect of the invention there is provided a cannula insertion system to insert a cannula into an arm of a patient, comprising:
a cannula insertion device configured to insert the cannula into the body part of the patient,
an arm support for supporting the arm during insertion of the cannula, and
a device arranged to pull the skin of the arm away from an insertion target area at opposite sides of the arm to stabilize the arm and/or stretch the skin at the insertion target area.

The device to pull the skin away from the insertion target area may be integrated in the arm support and may for example include a first pulling element and a second pulling element, for example a first pin and a second pin arranged at opposite sides of the arm. Downwards movement of the first and second pulling elements, e.g. movement of the pulling elements into the arm support when pushed against the arm may result in a pulling force on the skin. Tightening of the skin at the insertion target area by pulling the skin away from the insertion target area may prevent roll up of the skin during cannula insertion (e.g. with loose elderly skin) and/or to prevent rolling veins.

The first pulling element and the second pulling element may also be tactile feedback elements that provide tactile feedback to the patient with respect to the positioning of the body part.

According to yet another aspect of the invention there is provided a cannula insertion system for inserting a cannula into a body part of a patient, comprising:
a cannula insertion device configured to insert the cannula into the body part of the patient,
a body part support configured to support the body part during insertion of the cannula, and
an indicator device configured to guide positioning of the body part towards a desired position of the body part on the body part support.

The cannula insertion system may be an automatic or autonomous cannula insertion system. The automatic or autonomous cannula insertion system may also be configured as a biopsy device, a surgery robot, a laparoscopic robot, or any other system that may use an autonomous cannula insertion system in a body part of a patient.

Further characteristics and advantages of the cannula insertion system of the invention will now be explained by description of an embodiment of the invention, whereby reference is made to the appended drawings, in which:

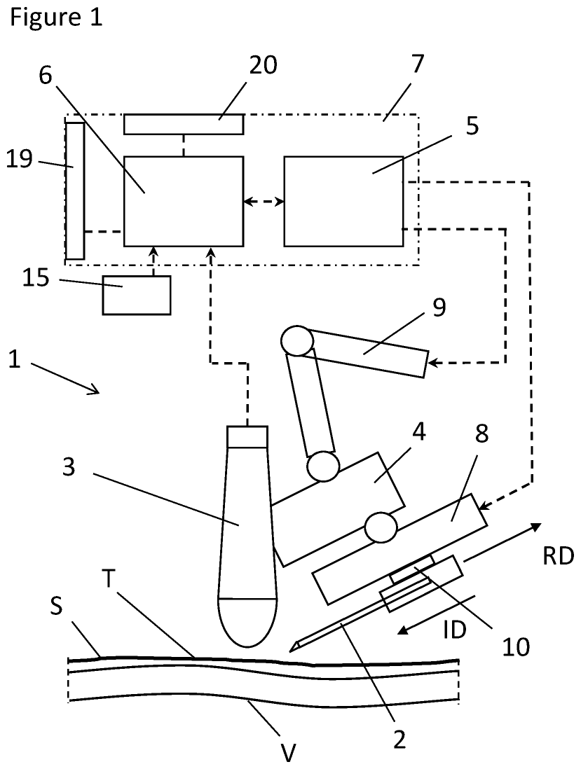

FIG. 1 shows schematically a first part of a cannula insertion system according to an embodiment of the invention comprising an end-effector supporting a contact sensor and a cannula insertion device;

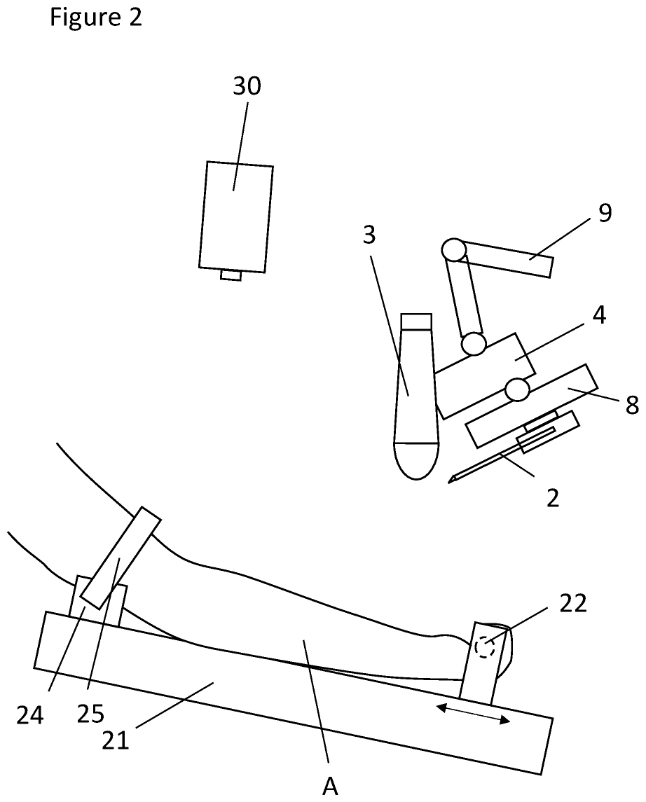

FIG. 2 shows schematically a part of the cannula insertion system comprising a light projection device according to an embodiment of the invention;

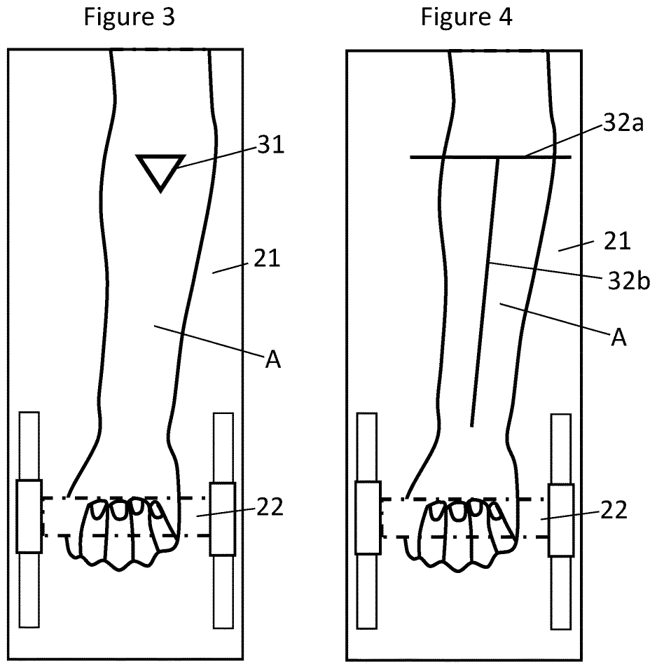

FIG. 3 shows schematically a first embodiment of a projected indicator;

FIG. 4 shows schematically a second embodiment of a projected indicator;

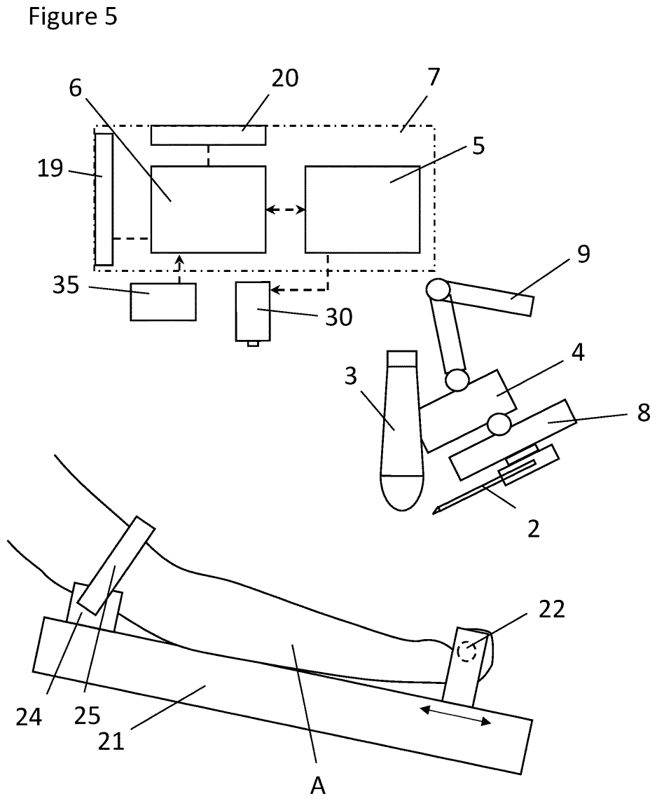

FIG. 5 shows schematically a part of the cannula insertion system comprising a light projection device according to an embodiment of the invention;

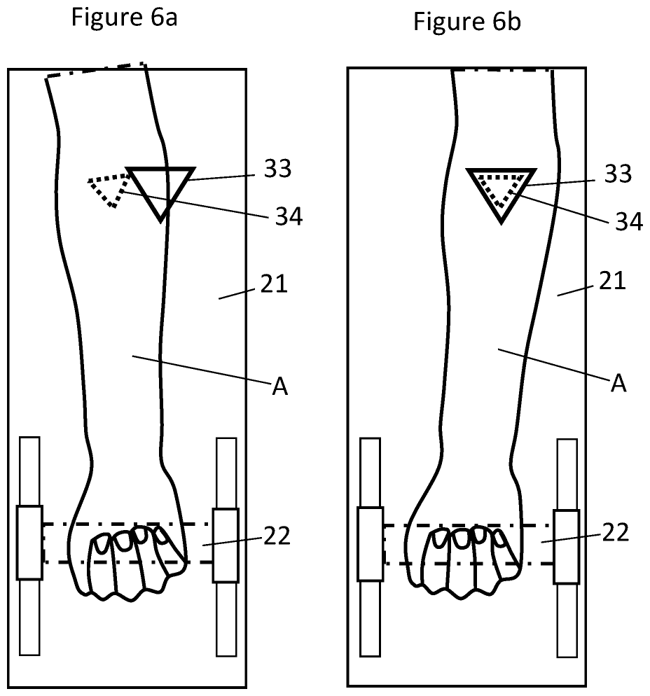

FIGS. 6a and 6b show a third embodiment of a projected indicator;

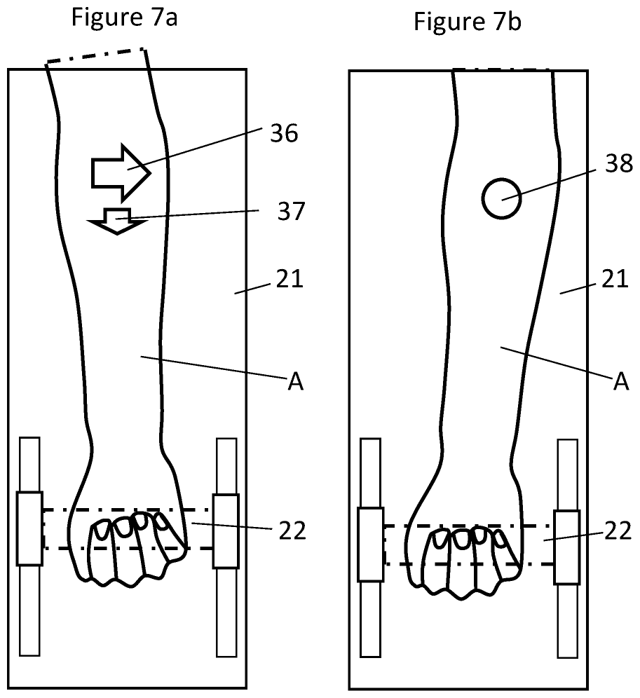

FIGS. 7a and 7b show a fourth embodiment of a projected indicator;

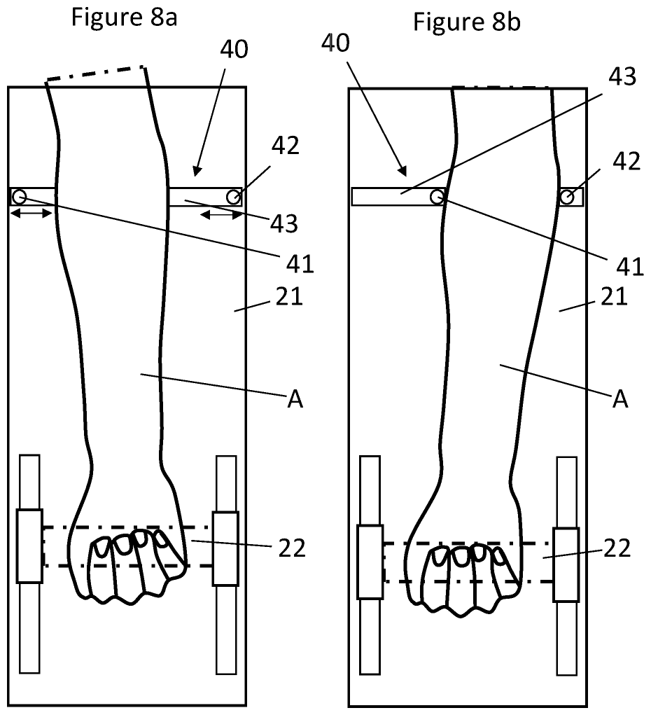

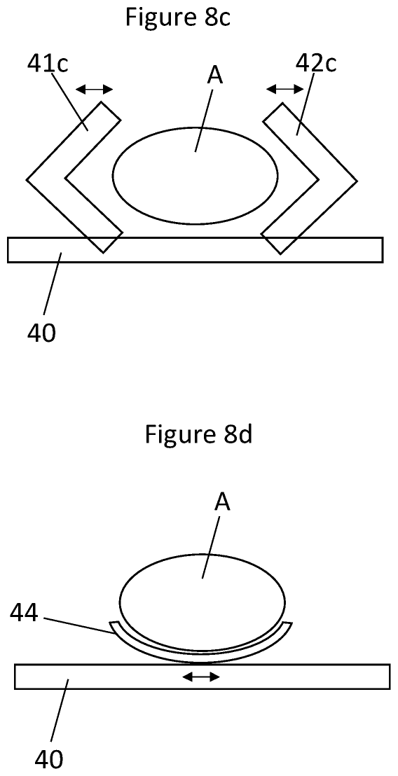

FIGS. 8a and 8b show an alternative embodiment of an indicator device for a cannula insertion system;

FIGS. 8c and 8d show alternative embodiments of a tactile feedback device in an indicator device for a cannula insertion system; and

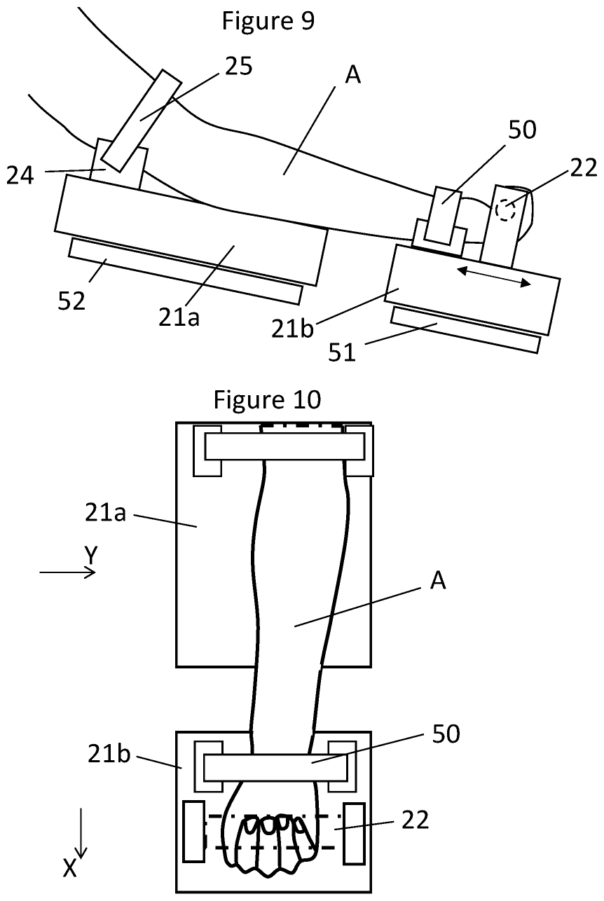

FIGS. 9 and 10 show another alternative embodiment of an indicator device for a cannula insertion system.

FIG. 1 shows schematically a cannula insertion system, generally denoted by reference numeral 1. Further details of the cannula insertion system are for example described in WO2021140116, the contents of which is herein incorporated by reference.

The cannula insertion system 1 is configured to autonomously insert a cannula 2, for example a needle into a blood vessel V of a patient. The patient may be any human or animal that uses the cannula insertion system 1 to insert a cannula into the body of the patient. The cannula insertion system 1 as shown in FIG. 1 is in particular configured to autonomously draw blood from a blood vessel. In alternative embodiments, the cannula insertion system 1 may be configured to arrange a cannula in a blood vessel V for intravenous medication and/or infusions.

To draw blood from the blood vessel V, the cannula insertion system 1 may be arranged to determine a location of a blood vessel underneath the skin S of the patient, insert a cannula 2 into the blood vessel V and draw blood from the blood vessel V without direct interaction of an operator of the cannula insertion system 1.

The cannula insertion system 1, shown in FIG. 1, comprises an ultrasound transducer 3 to obtain one or more sensor signals that are representative for the location of a blood vessel V in the patient. The ultrasound transducer 3 is a contact sensor. During use, contact between the ultrasound transducer 3 and the skin S enables the acquisition of relevant data with respect to the location of the blood vessel V. The ultrasound transducer may be guided along the skin S of the patient in a target area T. This target area T is an area of the skin S underneath which the presence of a blood vessel V suitable for insertion of a cannula 2 is expected and which is examined by the ultrasound transducer 3. A control device 5 controls the position of the ultrasound transducer 3.

The target area T may be determined by obtaining images of the skin of patient, for example using NIR (near infrared), infrared and/or visible light sensors 15, and determining on the basis of the images an area in which it is likely that a blood vessel suitable for insertion of a cannula will be found. The cannula insertion system 1 has multiple fixed sensors, i.e. sensors mounted at a fixed location, such as sensor 15. These multiple fixed sensors may be used to determine the target area, i.e. an approximate location of a blood vessel that can be used for insertion of the cannula 2. In an alternative embodiment, one or more of these NIR or visible light sensors may be arranged on a movable end-effector 4.

In addition, or as an alternative the fixed sensors 15 may be used to determine a location of the elbow pit of the arm and/or the shape of the arm. The sensors 15 could also be used to detect areas which should not be used for venipuncture, like birthmarks and wounds.

Once a target area T is determined, the ultrasound transducer 3 may be used to provide a sensor signal representative for a location of the blood vessel V within the target area T. The sensor signal is fed into a processing device 6 which is arranged to process the sensor signal. The control device 5 and the processing device 6 may be comprised in a single processer 7, such as a PC, or among a distributed network of multiple processors.

On the basis of the sensor signal provided by the ultrasound transducer 3, an image of the vein may be created by the processing device 6. This image may be based on multiple 2D images along a line in a certain direction, 3D coordinates and direction, 6D coordinates or may be a 3D image.

The processing device 6 determines on the basis of the sensor signal, or sensor signals, the location of a blood vessel V suitable for the insertion of the cannula 2. On the basis of this location, the processing device 6 may determine an insertion path for insertion of the cannula 2 into the blood vessel V. The processing device 6 may also locate nerves and prevent the cannula 2 from penetrating those nerves, when selecting an insertion path for the cannula 2.

If no suitable location for insertion of the cannula 2 can be found, the cannula insertion system 1 may request the patient to place the other arm in the cannula insertion system 1 (e.g., by displaying written and/or graphical instructions on a display device or other patient interface, providing audible instructions from a speaker device, etc.).

The cannula 2 is arranged on a cannula insertion device 8. The cannula 2 is held by a cannula holder 10. The cannula 2 can be taken out of the cannula holder to facilitate exchange of cannulas 2.

The cannula insertion device 8 is arranged to insert the cannula 2 in an insertion direction ID through the skin S and into the blood vessel V along the insertion path determined by the processing device 6. The cannula insertion device comprises a rotation actuator to adjust an insertion angle of the cannula 2 to align a longitudinal axis of the cannula 2 with the insertion direction ID. A linear actuator is provided to translate the cannula 2 along an insertion path in the insertion direction ID.

The insertion path of the cannula 2 may be adapted to anatomical structures in the arm of the patient. By adapting the position of the cannula insertion device 8 during insertion of the cannula 2, the cannula 2 may also be moved along a non-linear insertion path.

The cannula insertion device 8 may be provided with a force sensor with which the axial force exerted on the cannula 2 may be measured. By measuring the axial force exerted on the cannula 2, it can be determined when the wall of a vein is penetrated by the cannula 2. Further, this axial force on the cannula 2 may be compared with a maximum allowable axial force threshold value. Insertion of the cannula 2 may be stopped or at least the insertion speed may be lowered when this axial force exceeds this maximum allowable axial force threshold value.

The cannula insertion device 8 may comprise a safety device arranged to retract the cannula 2 or to release the cannula 2 from the cannula insertion device 8 when a force exerted on the cannula 2 in a direction perpendicular to the insertion direction ID results in exceeding a respective safety threshold value. The release of the cannula 2 from the cannula insertion device 8 may be a complete release or a release in a limited number of degrees of freedom, for example one or two rotation directions. The safety device may for example allow the cannula to freely rotate in the rotation direction in which a torque is exerted on the cannula when this torque exceeds a safety threshold value in order to follow an inadvertent movement of the patient.

The cannula insertion device 8 is supported by a positioning system 9, for example a robot arm, that is arranged to bring the cannula insertion device 8 in a position from which the cannula insertion device 8 may move the cannula 2 along the insertion path. The cannula insertion device 8 and the positioning system 9 are controlled by the control device 5.

In some cases, a vein positioned below the ultrasound transducer 3 may become displaced (e.g., laterally displaced) due to the pressure exerted by the ultrasound transducer 3. This may also be referred to as a ‘rolling vein’. When this occurs, this may be determined at least in part on the basis of the measurements of the ultrasound transducer 3. The cannula insertion system 1 may be configured to control the cannula insertion device 8 to actively follow the movement of the vein. In addition, or as an alternative, a mechanism may be provided to prevent displacement of the vein, for example by arranging two mechanical elements on the skin surface at opposite sides of the vein. Also, it is possible to abort the cannula insertion procedure when displacement of the vein is measured by the ultrasound transducer 3.

In the embodiment shown in FIG. 1, the ultrasound transducer 3 and the cannula insertion device 8 are mounted on a single end-effector 4. This ensures a fixed spatial relationship between the ultrasound transducer 3 and the cannula insertion device 8. The positioning system 9 is therefore used for positioning of both the ultrasound transducer 3 and the cannula insertion device 8. In an alternative embodiment, the ultrasound transducer 3 may be held in a fixed spatial relationship with the cannula 2, such that the tip of the cannula 2 can be tracked very accurately.

The cannula insertion system 1 comprises a patient or treatment identification device 20. The patient or treatment identification device 20 is configured to read an identification of the patient. Such identification may for example be an identity card, token or chip, a passport, a hospital identity card, token or chip or a letter with barcode, or an insurance card. The patient or treatment identification device 20 may comprise any suitable reader or scanner to read/scan the information provided by the identification of the patient. The patient or treatment identification device 20 may be connected with a central information system, such as an electronic patient file, a laboratory information system (LIS) or other system and may receive all data required for that patient for carrying out a cannula insertion procedure. After the identity of a patient has been determined, the identity of the patient may be confirmed, for example by providing an audible or readable output to the patient that may be confirmed by the patient.

The cannula insertion system 1 may comprise an emergency button that can be operated by the operator and/or the patient. When this emergency button is operated, the cannula insertion system will start an abort procedure, in which the cannula insertion process is safely stopped.

The cannula insertion system 1 may further comprise a patient interface, for example a display device 19 that can be seen by the patient.

The patient interface can be used to provide instructions to the patient, for example to reposition an arm or other body part, to place a different arm or other body part in the cannula insertion system 1, to provide information with respect to the cannula insertion procedure, such as the remaining time of the procedure, and/or display media, such as a video, that may distract the patient from the procedure. The patient interface may also be used for patient input. For example, the patient may be requested to respond to questions, such as to provide confirmation of the identity of the patient, the use of anticoagulation, etc.

Similarly, the cannula insertion system 1 may comprise an operator interface, such as a display device or communication device to provide relevant information with respect to the cannula insertion procedure to the operator. This operator interface may for example provide a sound or visual alarm system to inform the operator that the patient requests help. This operator interface may comprise a digital communication device that sends data to a remote device, e.g. a smart watch or tablet device.

FIG. 2 shows a part of the cannula insertion system 1 only partly shown in FIG. 1. FIG. 2 is used to show specific parts of a cannula insertion system 1 to describe/show different devices/elements of the cannula insertion system 1 and explain their function. In practice, the different devices/elements may be arranged close to each other and would be visible in the different Figures. Thus, some parts shown in one or more of the figures are deliberately not shown in other Figures. Also, not all devices/elements are drawn to scale with respect to the other devices/elements shown in the same or other Figures.

The cannula insertion system 1 comprises an arm support 21 on which the patient can place his arm. The arm support 21 may be adjustable in height based on, for example, the length/size of the patient. This height adjustment may be automatically or manually.

A hand grip 22 to be held by a hand of a patient is arranged on the arm support 21. The hand grip 22 can be movable, for example slidable, between a standby-position and an activated position (shown in FIG. 2). The hand grip 22 can include a member coupled to the arm support 21 and configured to be engaged (e.g., gripped or otherwise held) by the hand of a patient. For example, the member can be oriented approximately transverse to the arm support 21, and mounted at a height sufficiently spaced apart from the arm support 21 to a hand (e.g., palm) inserted between the member and the arm support 21. The hand grip 22 can include suitable ergonomic features, such as contoured finger grooves, padding, etc. to help make the hand grip 22 comfortable for a user to grasp. These ergonomic features may also be designed to ensure that the hand grip is held in a desired position of the hand, in particular with the hand palm faced upwards. In some embodiments, the hand grip 22 can include conformable material (e.g., padding, inflatable outer membrane) for the patient to actively squeeze, such as during a blood draw procedure after the cannula has been inserted.

The movable hand grip allows the arm support 21 to be adjusted to different arm lengths, while at the same time the elbow of the patient is arranged at substantially the same location. The hand grip 22 may be movable axially (e.g., generally in the direction of the length of the patient's arm) and/or transversely (e.g., generally orthogonal to the length of the patient's arm, from left side to right side of the patient's arm). A hand grip locking device may be provided to lock the hand grip 22 in a desired position.

In an embodiment, the hand grip 22 may be biased, for example by a biasing spring, to the stand-by position. At the start of the procedure the patient is requested to move the hand grip 22 to the activated position. In this position, the cannula insertion system 1 may carry out a cannula insertion procedure. When the hand grip 22 is moved back into the stand-by position, and the biasing spring pushes the hand grip 22 back to the stand-by position, the cannula insertion system 1 may be arranged to abort the cannula insertion procedure. Instead of the whole hand grip 22, it is also possible that only a part of the hand grip 22 is movable between a stand-by position and an activated position.

The cannula insertion system 1 may comprise a tourniquet device 24 to constrict and/or otherwise hold the upper arm of a patient during the cannula insertion procedure. The tourniquet device 24 comprises a strap or band 25 which may be manually or automatically tightened on the upper arm of a patient. The tourniquet device 24 can include one or more pressure sensors. The pressure with which the tourniquet device 24 is automatically tightened around the upper arm of the patient may depend at least in part on the patient itself, such as on the basis of the identification of the patient by the patient or treatment identification device 20 shown in FIG. 1. Additionally or alternatively, the pressure with which the tourniquet device 24 is tightened around the upper arm of the patient may be a predetermined pressure value (e.g., within a predetermined safe range of pressures). The actual pressure may be detected by the one or more pressure sensors.

The tourniquet device 24 may also comprise an inflatable cuff that is tightened around the upper arm of a patient. In such embodiment, the inflatable cuff may also be used to measure a blood pressure of the patient.

The arm support 21 may comprise a pressure sensor, proximity sensor, contact sensor (e.g., capacitive sensor) and/or other suitable sensor(s) to sense the presence of an arm on the arm support 21.

To correctly insert a cannula 2 into the arm A of a patient, it is of importance that the arm A of the patient is correctly positioned with respect to the arm support 21, with respect to the axial direction (e.g., along the length of the patient's arm) and in the transverse direction (e.g., orthogonal to the length of the patient's arm, from left to right). To indicate a desired correct position of the arm A with respect to the arm support 21, the cannula insertion system 1 comprises a light projection device 30. The light projection device 30 is arranged to project an indicator onto the arm and/or the arm support 21 that indicates a desired position of the arm A.

The light projection device 30 can be mounted at any suitable location, for example on a main frame of the cannula insertion system 1, the end-effector 4 or the positioning system 9. The light projection device 30 may for example be mounted on the same frame as the fixed sensors 15. The light projection device 30 comprises a light source, such as laser source or LED source, and may also comprise one or more optical elements such as lenses to project the indicator on the arm A of the patient. The light projection device 30 may be controlled by the control device 5 and the processing device 6 of the cannula insertion system 1. The light projection device 30 may, in some embodiments, only be activated at the beginning of the cannula insertion procedure during positioning of the arm A such that only during positioning of the arm A an indicator will be projected by the light projection device 30 on the arm A of the patient. For example, the light projection device 30 may be deactivated once the strap or band 25 of the tourniquet device 24 is tightened around the upper arm of the patient. However, in some embodiments the light projection device 30 may additionally or alternatively be activated during the cannula insertion procedure, while the cannula is inserted in the arm A, and/or during a cannula removal procedure (e.g., to help encourage the patient to maintain the arm A in the desired position during cannula insertion, and/or to help encourage the patient to remain substantially still to avoid disrupting the cannula or causing other complications when the cannula is in use or is being removed).

FIG. 3 shows a first example of an indicator 31 projected by the light projection device 30 onto the arm A of the patient. The indicator 31 is a triangle indicating a desired position of a cubital fossa of the arm. The patient may be requested, for example by an instruction on the display device 19 and/or by an audible instruction, to position the cubital fossa at or close to the triangle to properly position the arm A with respect to the arm support 21. As the cubital fossa of the arm and its direct surroundings is a preferred insertion location for a cannula 2 into the arm A of the patient, it is desirable that at least the cubital fossa is arranged at a desired location. This enables the cannula insertion system 1 to place the end effector 4 at an optimal start location with respect to the arm A of the patient to start a cannula insertion procedure.

FIG. 4 shows a second example of an indicator that can be used to position the arm A of the patient in a desired position with respect to the arm support 21. In this embodiment the indicator projected by the light projection device 30 onto the arm A of the patient comprises a first projected light line 32a indicating a desired position of an elbow crease or a superior border of the cubital fossa and a second projected light line 32b indicating a desired location of the longitudinal axis of the arm. The patient may be requested, for example by an instruction on the display device 19 and/or by an audible instruction, to position the elbow crease or superior border of the cubital fossa at or close to the first projected light line 32a and to position the middle of the arm aligned with the second projected light line 32b as shown in FIG. 4.

It will be clear that the indicator projected by the light projection device 30 onto the arm A of the patient may have a wide variety in shapes and sizes, such as contours, points and dots, to indicate the desired position of the arm A of the patient. In these embodiments, instructions can be provided to the patient how to position the arm A with respect to the indicators.

The cannula insertion system 1 may not be arranged to actively check whether the arm A is actually positioned in the desired position. The patient or operator of the cannula insertion system 1 may be requested to confirm that the arm is in the desired position, for example by a confirmation in an input device. After this confirmation, the tourniquet device 24 may be instructed to tighten the strap or band 25 around the arm A of the patient.

FIG. 5 shows an embodiment of a cannula insertion system 1 in which a sensor system 35 is arranged to determine an actual position of the arm A with respect to the arm support 21. This actual position of the arm A with respect to the arm support 21 may be determined directly or indirectly. Directly means that the sensor system 35 obtains images in which the position of the arm support 21 is compared with the position of the arm A. Indirectly means that the actual position of the arm A is not determined specifically with respect to the arm support 21 but with respect to any other reference of the cannula insertion system 1 of which the position with respect to the arm support 21 is known. The sensor system 35 may use sensors 15 as shown in FIG. 1. In addition, or as an alternative, other sensors may be provided to determine an actual position of the arm A.

The sensor system 35 may comprise a 2D camera system and/or a 3D camera system. Further, the sensor system 35 may be arranged to measure a 3D arm surface and/or may be configured to determine actual locations of predetermined features of the arm, such as the cubital fossa, the elbow crease, the wrist, etc.

The shape (e.g., pose and/or surface curvature) of the arm may be determined in order to determine the actual position of the arm.

In an embodiment, images obtained by one or more cameras may be used to create a 3D model of the arm surface. On the 3D surface model a strip of points that run on top of the upper surface of the 3D model along the center of the axial length of the patient's arm may be selected. Based on that collection of points, a straight line may be fitted between those points and the point that has the largest distance to the fitted line, indicates the location of the elbow crease and can be used to determine a location of the cubital fossa and/or another insertion target area.

In another method a complete 3D surface model of the upper surface of the arm can be used to find the shape (e.g., pose and/or curvature) of the arm. The 3D surface model can for example be fitted on a bone model or arm model to determine the curvature. On the basis of the determined curvature, a location of the cubital fossa and/or another insertion target area can be determined.

The control device 5 and the processing device 6 act together as a controller to process the data obtained by the sensor system 35 and to control the light projection device 30.

In the processing device 6 the actual position of the arm may be compared with the desired position of the arm with respect to the arm support 21 and it may be determined whether the arm A is positioned in the desired position.

The light projection device 30 is connected to the control device 5 and arranged to indicate, upon instruction of the control device 5, whether the arm A is in the desired position, or not, and/or to indicate the desired movement or direction of movement of the arm A from the actual position to the desired position.

In an embodiment, the light projection device 30 can be configured to indicate whether the arm is positioned in the desired position or not. For example, the light projection device 30 may project red light when the arm A is not in the correct position and green light when the arm is in the correct position.

In addition, or as an alternative, the light projection device 30 can be used to project an indicator that indicates the desired position, a movement to the desired position or a direction of movement of the arm from the actual position to the desired position.

FIGS. 6a and 6b show a first example in which the light projection device 30 is arranged to project a first indicator 33 representative for the desired position of the arm, in particular the desired position of the cubital fossa of the arm A of the patient and a second indicator 34 representative for the actual position of the arm A, in particular the actual position of the cubital fossa of the arm A of the patient. The first indicator 33 may correspond with the indicator 31 of FIG. 3.

The processing device 6 is arranged to determine the actual position of the arm, in particular the actual location of the cubital fossa of the arm A. The control device 5 may be configured to control the light projection device 30 to project the second indicator 34 that corresponds with the actual location of the cubital fossa of the arm A. The first indicator 33 and the second indicator 34 each have a triangular shape, wherein the triangular shape of the second indicator 34 is slightly smaller than the triangular shape of the first indicator 33 such that the second indicator 34 fits within the triangle of the first indicator 33.

FIG. 6a shows the first indicator 33 and the second indicator 34, wherein the first indicator 33 and the second indicator 34 are not aligned, i.e. the actual position of the cubital fossa does not correspond with the desired position of the cubital fossa. The patient may be requested to move his arm A to a position in which the second indicator 34 is aligned with the first indicator 33. In this embodiment, the second indicator 34 is aligned with the first indicator 33 when the triangular shape of the second indicator 34 is within the triangular shape of the first indicator 33.

FIG. 6b shows the first indicator 33 and the second indicator 34 in aligned position. Once the first indicator 33 and the second indicator 34 are in aligned position, the colour of one or both of the first and second indicator 33, 34 may be changed to confirm that the arm A has been moved into the desired position.

As the cannula insertion system 1 is able to check whether the arm A or at least the cubital fossa thereof is in the desired position, the cannula insertion system 1 can automatically continue the cannula insertion procedure, for example by instructing the tourniquet device 25 to tighten the strap or band 25 around the arm A of the patient.

The first indicator 33 and the second indicator 34 of FIGS. 6a and 6b are triangular shapes that are required to be aligned by bringing the second indicator 34 within the shape of the first indicator 33 in order to bring the arm A in the desired position. In alternative embodiments, other indicators may be used, wherein a first indicator indicates a desired position and a second indicator indicates an actual position and wherein alignment of the first indicator and the second indicator results in the desired position of the arm A on the arm support 21. These first and second indicators may have wide variety of shapes or other appearances, such as circles, beams, letters, logos, arrows, etc., and sizes, such as points, arm parts, or the complete under arm. Furthermore, the first and second indicators may have different appearances. For example, in an embodiment the first indicator may be a circle and the second indicator may be a triangle, and alignment can involve moving the arm A until the triangle is circumscribed by the circle. It is also possible that multiple first indicators to be aligned with multiple second indicators are used. Alignment can involve positioning the arm until the second indicator is positioned relative to the first indicator in any suitable designated relationship, such as one in which the first and second indicators at least partially overlap or touch. Alignment can, for example, involve moving the arm A until one indicator fits within the other indicator (e.g., the second indicator within the first indicator similar to that shown in FIGS. 6a and 6b, or vice versa), or until the indicators are overlaid (e.g., the first and second indicators are completely overlapping). As another example, each of the first and second indicators can include an alignment point (e.g., arrow point on an arrow, polygonal vertex), and alignment can involve positioning the arm until the respective alignment points on the first and second indicators touch.

FIGS. 7a and 7b show a second example of indicators that may be projected by the light projection device 30 based on a comparison between a desired position of the arm A and an actual position of the arm A.

FIG. 7a shows a first arrow-shaped indicator 36 indicating a desired transverse movement from the actual position of the arm A to the desired position of the arm A and a second arrow-shaped indicator 37 indicating a desired axial movement from the actual position of the arm A to the desired position of the arm A. The size (e.g., length) of the first arrow-shaped indicator 36 and the second arrow-shaped indicator 37 may indicate the distance of the desired movement in the transverse direction and the axial direction, respectively. Thus, the closer the actual position of the arm A to the desired position of the arm A, the smaller (e.g., shorter) the arrow-shaped indicators may be. Alternatively, color of one or both of the arrow-shaped indicators may change depending on how close the actual position of the arm A is to the desired position of the arm A (e.g., a color closer to red to indicate greater distance between the actual and desired positions, a color closer to green to indicate shorter distance between the actual and desired positions).

FIG. 7 b shows a circular shaped indicator 38 that indicates that the arm A is arranged in the desired position. This indicator 38 will be projected when the patient has properly moved the arm A from the actual position to the desired position as instructed by the first arrow-shaped indicator 36 and the second arrow-shaped indicator 37 as depicted in FIG. 7a. This circular shaped indicator 38 provides confirmation to the patient that the arm is positioned in the desired position. The circular shaped indicator 38 may also have a different colour than the first arrow-shaped indicator 36 and the second arrow-shaped indicator 37.

In addition to the projected indicators projected on the arm, other instructions or guidance may be provided to the patient to move or hold the arm A in the desired position. These instructions may for example be provided as audible instructions from an audio device (e.g., verbal, tone-based, etc.) and/or instructions displayed on the display device 19 of the cannula insertion system 1 (e.g., verbal, graphics-based, etc.). Confirmation that the arm is positioned in the desired position can additionally or alternatively be provided from the audio device (e.g., verbal, tone-based, etc.) and/or on the display device 19 (e.g., verbal, graphics-based).

It has been found that patients may be focused on their arm during the cannula insertion process. As a consequence, instructions provided on the display device 19 may not be noticed. Therefore, the light projection device of FIGS. 2 and 5 may also be used to project other instructions and/or information on the arm A of the patient. These instructions and/or this information, that may or may not be simultaneously be displayed on the display device 19 or otherwise provided to the patient is more likely to be noticed.

Instructions that may be displayed to the patient are for example instructions to keep the arm in a certain position and/or to twist the wrist to hold the hand grip in the desired way with the hand palm upwards. Information to be displayed may for example be the expected time needed to complete the cannula insertion procedure, or the phase of the procedure (insertion location detection, cannula insertion, blood withdrawal, bandage application, etc.).

It is further possible to display the projections on the body part, e.g. the arm, additionally on the display device 19. In this embodiment, the patient is provided with the same information on his arm and on the display. The display device 19 could be used to display a real-time video of the arm recorded with a camera.

FIGS. 8a and 8b shows a tactile feedback device 40 as an indicator device to indicate the desired position of an arm A on the arm support 21 and/or the desired movement of the arm A towards the desired position of the arm A on the arm support 21, and/or otherwise guide movement of the arm A toward the desired position of the arm A on the arm support 21. The tactile feedback device 40 comprises a first pin 41 and a second pin 42 that are each movable by actuators (not shown) along a guide 43 that extends transversely over the support surface of the arm support 21. The transverse direction of movement of the first pin 41 and the second pin 42 is indicated by double headed arrows. Alternatively, one of the first and second pins can be static, while the other of the first and second pins can be movable. One or both of the first and second pins may include padding or other conformable material on their outer surfaces, to help increase patient comfort when the pins contact the skin of the patient.

FIG. 8a shows the first pin 41 and the second pin 42 in an example start position. In this start position, the arm A can be moved onto the arm support 21 between the first pin 41 and the second pin 42. A sensor system, for example the sensors 15 of FIG. 1 or the sensor system 35 of FIG. 5, may be used to determine an actual position of the arm A with respect to the arm support 21.

A controller, for example the combination of control device 5 and processing device 6, may compare the actual position with the desired position of the arm A on the arm support 21 in order to determine whether the body part is in the desired position and/or to determine the desired movement of the body part from the actual position to the desired position,

The tactile feedback device 40 may be connected to the controller, for example the control device 5, to slowly move the first pin 41 and the second pin 42 to positions at opposite sides next to the arm A in the desired position. If the arm A is not yet in the desired position as shown in FIG. 8 a, one of the first pin 41 and the second pin 42 will contact the arm A and exert a light force on the arm surface as an indicator that the arm should be moved in a direction opposite to the location of the respective pin 41,42. For example in the position of FIG. 8a the first pin 41 will first come into contact with the arm to indicate that the arm should be moved towards the second pin 42. The first pin 41 or the second pin 42 may additionally or alternatively vibrate to indicate that the arm A should be moved. This tactile feedback of the first pin 41 and the second pin 42 may be continued until the arm A is positioned in the desired position on the arm support 21.

FIG. 8b shows the arm positioned in the desired position on the arm support 21. The first pin 41 and the second pin 42 may remain in this position during the cannula insertion procedure to ensure that the arm A is maintained in the desired position. It is also possible to move the first pin 41 and the second pin 42 back to the start position, for example when the tourniquet device 42 (not shown in FIGS. 8a and 8b) and the hand grip 22 provide sufficient support to maintain the arm A in the desired position.

The first pin 41 and/or the second pin 42 can be used to move the arm A to at least the desired transverse position. To move the arm axially to the desired axial position, the hand grip 22 may be used. The hand grip 22 may be provided with an actuator to move the hand grip 22 in the axial direction. When the processing device 6 determines that the arm A is not in the desired axial position, the hand grip 22 may be moved in the axial direction to press against the hand in order to indicate to the patient that the arm should be moved to follow the hand grip 22 in the indicated direction. Similar to the first pin 41 and the second pin 42 the hand grip may provide an additional signal, for example a vibration, to indicate that the movement of the hand grip 22 should be followed. This could also be done with additional instruction provided as an audible instruction, an instruction displayed on the display device 19 and/or projected on the arm with a light projection device 30.

Once the arm A is positioned in the desired axial position, the tactile feedback provided by the hand grip 22 may be stopped.

The first pin 41, the second pin 42 and the hand grip 22 are examples of tactile feedback elements that can be used in a tactile feedback device 40 to provide tactile feedback to the patient with respect to the positioning of the arm A. Other tactile feedback elements may also be used. The tactile feedback device 40 may be used in combination with a light projection device 30 to provide both tactile indicators and light indicators that facilitate the patient to position the arm A in a desired position on the arm support 21.

In an embodiment, the first pin 41 and the second pin 42 may be used, when placed against the arm A at opposite sides of the arm A, to pull the skin of the arm away from the insertion target area at the contact locations between the pins 41, 42 and the arm A, for example by movement of the pins 41, 42 into the plane of the drawings of FIGS. 8a and 8b. The first and/or second pins can include an adhesive or textured surface (e.g., ribs) to help engage the skin and pull the skin away from the insertion target area when the pins move in such a manner. This skin pulling may help to further stabilize the arm A and may stretch the skin at the target area of the cannula insertion. This stretching of the skin may also stabilize the position of veins within the arm and therewith reduced the movement of the veins during insertion of the cannula. The pins 41, 42 may be provided with friction increasing shapes or material to pull along the skin of the patient.

Other devices to pull the skin of the arm away from the insertion target area at opposite sides of the arm to stabilize the arm and/or stretch the skin at the insertion target area may also be provided.

While FIGS. 8a and 8b show a tactile feedback device 40 including pins, the tactile feedback device 40 can include one or more movable contacts having any suitable shape for contacting the arm of the patient and guiding its movement toward the desired position of the arm. For example, the tactile feedback device can include one or more lateral movable walls that contacts or receives a respective lateral side of the arm A of the patient. Such movable walls can for example be generally planar. As another example, such movable walls can include a lateral cradling surface (e. g ., with a L-shaped, V-shaped, C-shaped or other arcuate-like cross-section) that surrounds or receives at least a lateral portion of the arm A of the patient (e.g., contacts both upper and lower surfaces of a left or right side of the arm A), such as first contact 41c and second contact 42c shown in FIG. 8c. As yet another example, as shown in FIG. 8d, the tactile feedback device can include a movable contact including a cradle 44 configured to receive at least an underside portion (e.g., elbow) of the patient arm A.

FIGS. 9 and 10 show another embodiment to position the arm A of a patient in a desired position. In this embodiment the arm support is split in an elbow support 21a and a wrist support 21b. The wrist support 21b is independently movable with respect to the elbow support 21a in at least an axial direction X and the elbow support 21a is movable with respect to the wrist support in a transverse direction Y. The wrist support 21b comprises a wrist fixation device 50 to fixate the wrist on the wrist support 21b. Correspondingly, the tourniquet device 24 can be used to fix the elbow on the elbow support 21a. A wrist support actuation device 51 is provided to move the wrist support 21b in axial direction X. The movement of the wrist support 21b with respect to the elbow support 21a works as an indicator device to the patient as the patient will be required to follow the movement of the wrist support 21b. An elbow support actuation device 52 is provided to move the elbow support 21a in transverse direction Y with respect to the wrist support 21b. The movement of the elbow support 21a with respect to the wrist support 21b works as an indicator device to the patient as the patient will be required to follow the movement of the elbow support 21a.

To properly position an arm A on the arm support the following sequence can be followed. As a first step the patient will move the arm A into the cannula insertion system 1 through the openings of the straps or band of the tourniquet device 24 and the wrist fixation device 50. When it is detected that the hand grip 22 is held by the hand of the patient, the strap or band of the wrist fixation device 50 may tighten around the wrist of the patient. On the basis of the measurements of a sensor system, for example the sensors 15 or the sensor system 35 described above, the processing device 6 can determine whether the arm is in the correct axial position. If the arm A is not yet in the correct axial position a gentle movement of the wrist support 21b actuated by the wrist support actuation device 51 with respect to the elbow support 21a may move the arm A forwards or backwards in the X direction to a desired position.

Once the desired position in axial direction has been reached, the band or strap 25 of the tourniquet device 24 may be tightened around the upper arm of the patient to fixate the arm A also with respect to the elbow support 21a. Now, on the basis of the measurements of a sensor system, the processing device 6 can determine whether the arm A is in the correct transverse position. If the arm A is not yet in the correct transverse position a gentle movement of the elbow support 21a actuated by the elbow support actuation device 52 may move the elbow sidewards with respect to the wrist in the (positive or negative) Y direction to the desired position of the arm A with respect to the arm support formed by the elbow support 21a and the wrist support 21b.

By following this subsequential movement in X direction and Y direction, it is first ensured that the arm A is positioned in the correct axial position before the arm A is fixated both at the wrist and the upper arm. However, in some embodiments, the movement of the elbow support 21a and the wrist support 21b can position the arm A first in the correct transverse position in the Y direction, followed by positioning the arm A in the correct axial position in the X direction.

Hereinabove, different indicator devices to indicate a desired position of the arm A of a patient with respect to an arm support and/or a desired movement or direction of movement of the body part towards the desired position of the body part on the body part support have been described for a cannula insertion system. The cannula insertion system may also be used to insert a cannula in other body part, such as a leg of a patient. The indicator devices may also be adapted to be used for positioning of such other body part.

1. A cannula insertion system for inserting a cannula into a body part of a patient, comprising:
a cannula insertion device configured to insert the cannula into the body part of the patient,
a body part support for supporting the body part during insertion of the cannula, and
an indicator device to indicate a desired position of the body part on the body part support and/or a desired movement or direction of movement of the body part towards the desired position of the body part on the body part support.

2. The cannula insertion system of claim 1, wherein the indicator device comprises a light projection device arranged to project an indicator onto the body part and/or the body part support.

3. The cannula insertion system of claim 2, wherein the body part is an arm and the indicator is a projected light indicator indicating a desired position of a cubital fossa of arm.

4. The cannula insertion system of claim 2, wherein the body part is an arm and the indicator is a first projected light line indicating a desired position of an elbow crease or a superior border of the cubital fossa and/or a second projected light line indicating a desired location of the longitudinal axis of the arm.

5. The cannula insertion system of claim 1, wherein the cannula insertion system further comprises:
a sensor system arranged to determine an actual position of the body part with respect to the body part support or another reference of the cannula insertion system, and
a controller to compare the actual position with the desired position of the body part on the body part support in order to determine whether the body part is in the desired position and/or to determine the desired movement of the body part from the actual position to the desired position,
wherein the indicator device is connected to the controller and arranged to indicate whether the body part is in the desired position, or not, and/or to indicate the desired movement or direction of movement of the body part from the actual position to the desired position.

6. The cannula insertion system of claim 2, wherein the light projection device is arranged to project a first indicator representative for the desired position of the body part and a second indicator representative for the actual position of the body part, such that the body part can be brought into the desired position by predetermined alignment of the second indicator with respect to the first indicator.

7. The cannula insertion system of claim 5, wherein the sensor system comprises a 2D camera system and/or a 3D camera system.

8. The cannula insertion system of claim 5, wherein the sensor system is arranged to measure a 3D body part surface and/or to determine actual locations of predetermined features of the body part.

9. The cannula insertion system of claim 5, wherein the indicator device comprises a light projection device arranged to project an indicator onto the body part, wherein the light projection device is configured to project at least two different colors, wherein the controller instructs the light projection device to project a first color of the at least two different colors when the body part is arranged in the desired position on the body part support and a second color of the at least two different colors when the body part is not arranged in the desired position on the body part support.

10. The cannula insertion system of claim 1, wherein the body part is an arm and the body part support is an arm support.

11. The cannula insertion system of claim 10, further comprising a controller, wherein the controller is arranged to determine a shape, for example a pose and/or surface curvature of the arm of the patient.

12. The cannula insertion system of claim 10, wherein the cannula insertion system comprises a hand grip to be held by the hand of the arm supported on the arm support.

13. The cannula insertion system of claim 12, wherein the hand grip is designed such that in the desired position of the hand the hand palm is faced upwards.

14. The cannula insertion system of claim 1, wherein the indicator device comprises a tactile feedback device to indicate the desired position of the body part on the body part support and/or the desired movement of the body part towards the desired position of the body part on the body part support.

15. The cannula insertion system of claim 1, wherein the cannula insertion system comprises a body part fixation device to fixate the body part on the body part support.

16. The cannula insertion system of claim 15, wherein the body part support comprises an actuation device to actuate the body part fixation device to move the fixated body part into a desired position.

17. The cannula insertion system of claim 1, wherein the cannula insertion device comprises a display device to display instructions to the patient.

18. The cannula insertion system of claim 1, wherein the cannula insertion system comprises a light projection device arranged to project instructions and/or information on the body part.

19. A cannula insertion system to insert a cannula into a body part of a patient, comprising:
a cannula insertion device configured to insert the cannula into the body part of the patient,
a body part support for supporting the body part during insertion of the cannula, and
a light projection device arranged to project instructions and/or information on the body part.
